(19) United States
(12) Patent Application Publication
(10) Pub. No.: US 2014/0221928 A1
(43) Pub. Date: Aug. 7, 2014

(54) DEVICE FOR SUCTION OF LIQUIDS AND/OR PARTICLES FROM BODY ORIFICES

(71) Applicants: Lena Ginzinger, Baden-Baden (DE); Peter Harlacher, Buhl (DE)

(72) Inventors: Lena Ginzinger, Baden-Baden (DE); Peter Harlacher, Buhl (DE)

(73) Assignee: Maquet GmbH & CO. KG, Rastatt (DE)

(21) Appl. No.: 14/234,862

(22) PCT Filed: Jul. 27, 2012
(86) PCT No.: PCT/EP2012/064803

§ 371 Date: Apr. 4, 2014

(30) Foreign Application Priority Data
Jul. 27, 2011 (DE) 10 2011 052 196.8

(51) Int. Cl. A61M 1/00

(52) U.S. Cl. CPC A61M 1/0033; A61M 1/0047; A61M 2205/3344; A61M 2205/3351
USPC 604/151

(57) ABSTRACT

The invention relates to a device for suction of liquids and/or particles from body orifices, wherein the device comprises a suction channel (16) through which the liquid and/or particles can be aspirated and a vacuum pump (14) for generating an underpressure in the suction channel (16). The suction channel (16) is provided with an opening for supplying additional air for adjusting the underpressure, wherein the volume flow of this additional air is adjustable via an additional air adjustment unit (24). Further, the device (10) has a manually operable adjustment unit (26) for adjusting the additional air adjustment unit (24), wherein the adjustment of the adjustment unit (26) in each case by a same preset adjustment way effects in each case the same change in the underpressure.

### CROSS REFERENCE TO RELATED APPLICATIONS

This application is entitled to the benefit of and incorporates by reference subject matter disclosed in International Patent Application No. PCT/EP2012/064803 filed on Jul. 27, 2012 and German Patent Application No. 10 2011 052 196.8 filed Jul. 27, 2011.

### TECHNICAL FIELD

The invention relates to a device for suction of liquids and/or particles from body orifices, wherein the device comprises a suction channel through which the liquid and/or the particles can be extracted. Further, the device comprises a vacuum pump for generating an underpressure in the suction channel, wherein as a consequence of this underpressure the liquid and/or the particles are sucked and wherein the suction channel is provided with an opening for supplying additional air for adjusting the underpressure. Further, the device is provided with an additional air adjustment unit for adjusting the volume flow of the additional air supplied to the suction channel as well as a manually operable adjustment unit for adjusting the additional air adjustment unit.

### BACKGROUND

The device is in particular used in hospitals, doctors' offices, nursing homes and/or in home care, for example to remove blood or other liquids, inclusive the particles or bone fragments contained therein, during operations from natural and/or artificial body orifices. The device is also commonly referred to as secretion fluid suction device.

In order to adjust the suction power of the suction device to the respective application, it is possible to control the suction power by varying the underpressure. For this purpose, additional air is supplied to the suction channel, wherein in case of known suction devices the amount of the supplied additional air may be adjusted via a needle valve. For manually adjusting this needle valve at a user interface of the suction device a rotary knob is provided, wherein by means of turning this knob the needle valve is adjusted and thus the volume flow of the additional air is varied as well. Due to the disproportional characteristic curve of the needle valve and the non-linear functional relationship between the supplied volume flow of additional air and the resulting underpressure at a relatively low underpressure for the same variation of the underpressure the rotary knob must be turned by a substantially larger angular range than is necessary when adjusting the same variation in the underpressure in a high underpressure range.

Here, the problem is that only little ease of operation can be achieved, as the operator must always watch very closely via the vacuum gauge for displaying the current underpressure which underpressure is presently prevailing, thus making an intuitive operation difficult.

### SUMMARY

It is the object of the invention to specify a device for suction of liquids and/or particles from body orifices, the suction power of which is easily adjustable.

This object is solved by a device having the features of claim 1. Advantageous developments of the invention are specified in the dependent claims.

According to the invention the adjustment unit and the additional air adjustment unit are designed such that at least in the range between a lower limit value of the underpressure and an upper limit value of the underpressure the adjustment of the adjustment unit in each case by a same preset adjustment way effects in each case the same variation in the underpressure. By this, an especially simple operation of the device is achieved during adjustment of the underpressure, as for the same change in the volume flow of the additional air, and thus for the same change in the underpressure of the underpressure used for suction, the operator has to adjust the adjustment unit in each case by the same adjustment way.

The underpressure prevailing in the suction channel is that underpressure resulting when the opening of the suction channel through which the liquid and/or the particles are sucked is closed. Consequently, the underpressure prevailing in the suction channel is the maximum underpressure that can be generated at the respective position of the additional air adjustment unit.

The lower limit value of the underpressure is that minimum underpressure that can be generated by means of the device. Correspondingly, the upper limit value of the underpressure specifies that maximum underpressure that can be generated by means of the device. In particular, the upper limit value has a value between −70 kPa and −90 kPa, preferably of approximately −80 kPa. The lower limit value has in particular a value of 0 kPa. The respective underpressures relate preferably to the respective prevailing atmospheric pressure.

The adjustment unit is in particular a rotatable knob or ring, so that the adjustment way corresponds to one angular rotation. Thus, to change the underpressure by a same value in each case the same angle of rotation must be adjusted. Alternatively, the adjustment unit can be designed such that it comprises a linearly movable slider, wherein in this case for the same change in the underpressure the slider must be moved in each case by the same way.

In particular, the additional air adjustment unit can be designed such that by means of it the opening of the suction channel, through which the additional air is supplied, is closable.

In this case the flow cross-section of the volume flow of the additional air is changed in that this opening is closed partly or entirely. The flow cross-section is that cross-section that is available to the volume flow of the additional air during supply to the suction channel. Alternatively, the additional air adjustment unit can be arranged such that by means of it the cross-section of an additional air channel, through which the additional air is supplied to the opening, is closable partly or entirely.

Due to a change in the flow cross-section of the volume flow of the additional air also the amount of additional air supplied per time unit and thus the underpressure prevailing in the suction channel change. This in turn has the result that the suction power with which the liquid and/or the particles are sucked from the body orifice, varies correspondingly and is thus easily adjustable.

In a preferred embodiment the adjustment unit can be adjusted stepwise, wherein at least in the range between the lower limit value and the upper limit value the adjustment of the adjustment unit in each case by one step effects in each case the same change in the underpressure. For this purpose, the adjustment unit is particularly designed such that it engages according to the step. Alternatively, the adjustment unit can be continuously adjustable.

The additional air adjustment unit is in particular provided with a valve by means of which the flow cross-section of the volume flow of the additional air is adjustable. Thus, without a complex coupling between the adjustment unit and the valve in a simple manner a corresponding linear relationship between the adjustment way of the adjustment unit and the change of the underpressure can be achieved.

Further, the additional air adjustment unit may comprise an aperture for closing at least one portion of an additional air channel for supplying the additional air and/or for closing at least one portion of the opening for supplying the additional air. Via such an aperture it is particularly easy to change the underpressure in synchronization with the adjustment way of the adjustment unit. Here, the additional air channel and/or the opening have in particular a triangular cross-section, wherein the additional air adjustment unit comprises a helical aperture, via which the cross-section is closable partly or entirely. Thus, in case of turning the aperture by the same angle, the cross-section is in each case enlarged or reduced such that the volume flow of the additional air changes itself such that the desired linear synchronous coupling between the adjustment unit and the underpressure prevailing in the suction channel will be achieved.

In a preferred embodiment, a manometer is provided for displaying the respective current underpressure, so that the operator can monitor in a simple manner which underpressure is prevailing currently in the suction channel. In particular, by this it can be avoided to impair the patient's health due to a too high underpressure.

The manometer has in particular a circular-sectored scale and a pointer that is rotatably arranged around the centre of the circular scale for displaying the current underpressure on the scale. The adjustment unit is provided with a ring, the centre of which coincides with the centre of the scale and which is rotatably arranged around its centre for adjusting the underpressure. On the ring at least one marking is provided, wherein the adjustment unit is synchronized with the manometer such that at least in the range between the lower limit value and the upper limit value in each case that underpressure in the suction channel and thus also on the manometer adjusts itself that is displayed by the marking of the ring on the scale. Thus, a dual use of the scale is achieved, namely on the one hand for displaying the current underpressure by the manometer and on the other hand for adjusting the desired underpressure via the adjustment unit by the operator. In particular, by this it is possible for the operator to adjust the desired underpressure in an especially simple manner. Unlike as is the case with known suction devices with a needle valve, the operator does not have to adjust the rotary knob of the suction device in a time-consuming manner by means of repeated trying as long as eventually the desired underpressure has adjusted itself.

Preferably, at least a part of the suction channel is formed by means of a suction hose that can easily be introduced into the body orifice. In particular, an electric motor is provided for driving the vacuum pump.

The marking of the ring is preferably designed in form of a deepening in the surface of the ring, so that this deepening does not only serve as a marking, but the ring can also be rotated manually in an easy manner by means of placing a finger into this deepening. By this, an especially simple operation is achieved.

Additionally or alternatively, the marking is done by using a marking in a colour that is different from the colour of the ring.

In particular, the ring is designed such that it is rotatably arranged by 210°. Correspondingly, the scale is also designed such that between the lower limit value and the upper limit value an angle between 210° is arranged.

### BRIEF DESCRIPTION OF THE DRAWINGS

Further features and advantages of the invention result from the following description which in connection with the enclosed Figures explains the invention in more detail with reference to embodiments.

FIG. 1 is a schematic view of a device for suction of liquids and/or particles from body orifices,

FIG. 2 is a schematic view of an operating unit of the device according to FIG. 1,

FIG. 3 is a schematic perspective view of an additional air adjustment unit of the device according to FIGS. 1 and 2,

FIG. 4 is a top view of the additional air adjustment unit according to FIG. 3 in a first position,

FIG. 5 is a top view of the additional air adjustment unit according to FIG. 3 in a second position, and

FIG. 6 is a top view of the additional air adjustment unit according to FIG. 3 in a third position.

### DETAILED DESCRIPTION

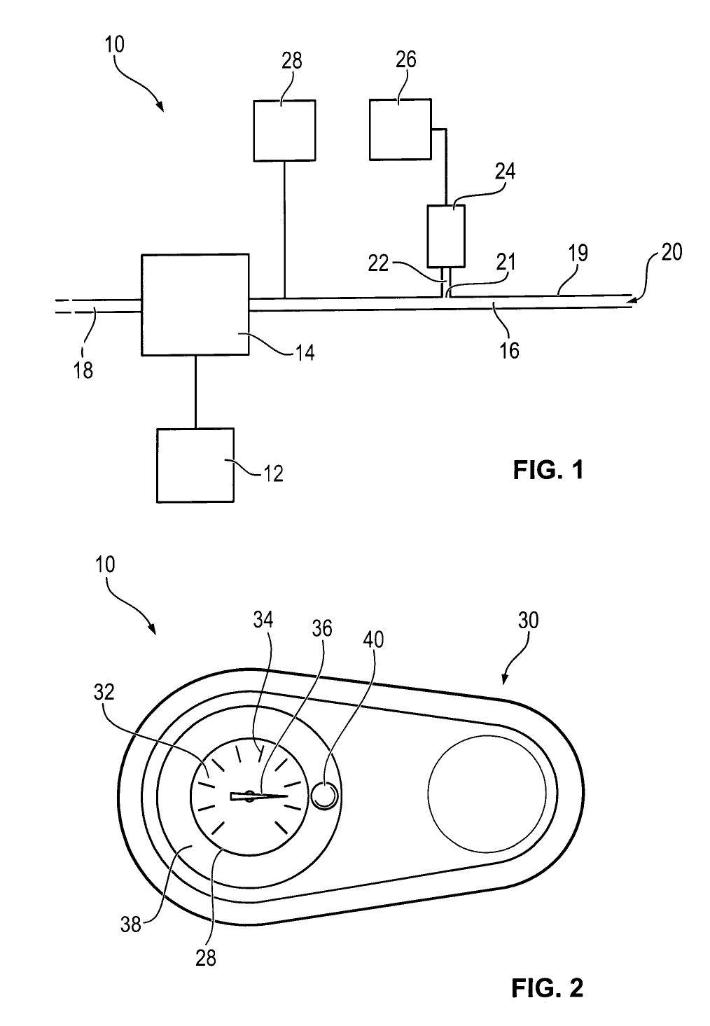

In FIG. 1 a schematic view of a device 10 for suction of liquids and/or particles from body orifices is provided that is normally used in the medical field, for example in hospitals, doctors' offices, nursing homes and/or in home care and that is commonly known as secretion fluid suction device.

The device 10 comprises a pump (compressor) 14 that is driven via an electric motor 12, wherein via the pump in a suction channel 16 an underpressure can be generated via which the fluid and/or the particles are extracted from the body orifices. Here, power supply of the motor can take place via a power supply unit that can be connected to the power supply system or via accumulators, so that the device 10 may be utilized optionally for stationary use as well as for mobile use. Further, an exhaust air channel 18 is provided via which the air extracted by the pump via the suction channel 16 is discharged again, after the fluid and/or the particles have been removed from the channel and have been filled into a not-illustrated container.

At least a portion of the suction channel 16 is formed by a suction hose 19 that at its end facing away from the pump 14 is provided with a suction opening 20, through which the liquid and/or the particles are aspirated. Such a suction hose 19 can be easily introduced into the corresponding body orifice, so that the liquid and/or the particles can be extracted easily and a simple, injury-free and comfortable handling is possible.

In the suction channel 16 an additional air opening 21 is provided via which additional air supplied by means of an additional air channel 22 can be supplied to the suction channel 16. The volume flow of this supplied additional air is adjustable via an additional air adjustment unit 24. The additional air unit 24 is described in more detail in connection with FIGS. 3 to 6.

Via the volume flow of the additional air the underpressure prevailing in the suction channel 16 and thus the suction power of the device 10 can be adjusted. The greater the supplied volume flow of the additional air, the lower the underpressure.

Moreover, a manometer 28 is provided for displaying each underpressure prevailing in the suction channel 16, so that this underpressure can be monitored easily by the operator. The additional air adjustment unit 24 adjusts the volume flow of the additional air by varying the cross-section of the additional air channel 22 and/or the opening 21 being provided for the volume flow of the additional air.

FIG. 2 is a schematic view of an operating panel 30 of the device 10. The manometer 28 is provided with a display area 32 in which a circular-sectored scale 34 is arranged. On this scale 34 via a pointer 36 the respective underpressure prevailing in the suction channel 16 is displayed. The scale 34 extends in particular over an angular range of 210°. Alternatively, the scale 34 can be formed over a greater or smaller angular range, in particular the scale 34 may be circular. The maximum underpressure that can be adjusted by means of the device is in particular −80 kPa and is also designated as upper limit value. The minimum underpressure that can be adjusted is in particular 0 kPa, i.e. that the atmospheric pressure prevails and thus there is no underpressure. Correspondingly, the scale 34 extends in particular at least from 0 kPa to −80 kPa.

The adjustment unit 26 is provided with a ring 38 that is arranged concentrically to the scale 34; i.e. that the centre of the ring 38 coincides with that point about which the pointer 36 is rotatably arranged. The ring 38 is also rotatable around this centre. On the surface of the ring 38 a recess 40 is provided via which the ring 38 can easily be turned by the operator and via which a marking is formed. Further, this recess 40 has a colour that is different from the colour of the surface of the ring 38 in which the recess 40 is formed.

The ring 38 and the additional air adjustment unit 24 are synchronized to each other such that during turning of the ring 38 in each case by the same angle the cross-section of the volume flow of the additional air released by the additional air adjustment unit 24 changes in such a way that there is a linear connection between the underpressure and the angle of rotation of the adjustment unit 26, in particular of the ring 38. Furthermore, the manometer 28 and the ring 38 are matched to each other such that in each case that underpressure adjusts itself that is displayed by the deepening 40 of the ring on the scale 34 of the manometer 28, so that the desired underpressure can easily be adjusted by the operator. By means of synchronizing the ring 38 and the manometer 28 a particularly simple intuitive operation of the device 10 is achieved. Further, unlike is the case with known suction devices, for adjustment of the underpressure the suction opening 20 does not have to be closed.

In particular, the additional air adjustment unit 24 is provided with an aperture and/or a slider that during turning of the ring 38 partly or entirely closes the cross-section of the opening 21 and/or the cross-section of the additional air channel 22 and thus adjusts the flow cross-section being provided for the volume flow of the additional air. Here, during turning of the ring 38 by 1° the slider is in each case adjusted by the same adjustment way, so that in case of a triangular cross-section of the opening 21 or respectively the additional air channel 22 and a helical cross-section of the aperture or respectively the slider a linear connection between the angle of rotation of the ring 38 and the underpressure and thus the suction power of the device 10 occurs.

Alternatively, instead of a slider or an aperture also a valve with a corresponding characteristic curve may be used.

Further, alternatively it is possible to provide a rotary knob instead of a ring 38, via which the respective underpressure is adjustable. This rotary knob is in particular arranged adjacent to the manometer 28, but not arranged concentrically. In this case there is also the advantage that for adjusting the underpressure by the same value in each case the same turning of the rotary knob is required and thus a simple operation is achieved. However, it is not possible to adjust the desired underpressure via the scale 34 of the manometer 28.

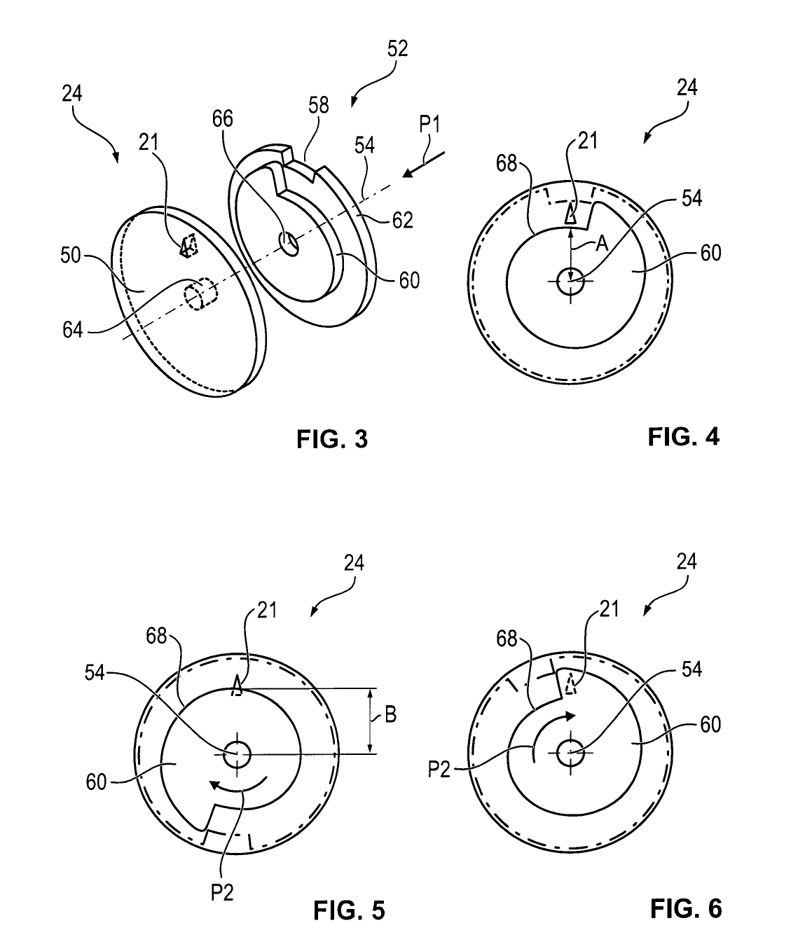

FIG. 3 is a schematic perspective view of the additional air adjustment unit 24 in an exploded view. FIG. 4 is a top view of the additional air adjustment unit 24 according to FIG. 3 in a first position with viewing direction in direction of the arrow P1 according to FIG. 3.

The additional air adjustment unit 24 comprises a stationary disc 50 in which the opening 21, through which the additional air can be introduced into the suction channel 18, is formed. The opening 21 has a triangular cross-section, wherein this triangle is in particular an acute-angled, equal-sided triangle.

Further, the additional air adjustment unit 24 comprises a rotatably arranged rotary unit 52 that comprises an aperture 60 as well as a disc 62 that is firmly connected with the aperture 60. The aperture 60 and the disc 62 are in particular formed in one piece.

The aperture 60 is in particular helically formed, i.e. in form of a cam. Due to this, the contour 68 of the aperture has in particular a spiral form.

The disc 50 and the rotary unit 52 are in particular arranged concentrically to each other, so that they are rotatably arranged around a common centre axis 54. For this purpose, the disc 50 is in particular provided with a bolt 64 which engages in a complementarily formed recess 66 of the rotary unit 52.

In the disc 62 of the rotary unit 52 a recess 58 is provided, so that the additional air flowing through the opening 21 can flow through this recess 58.

The helical form of the aperture 60 is formed such that the distance A of its contour 68 to the centre axis 54 varies depending on the angle of rotation of the aperture 60. In case of the first position shown in FIG. 4, the aperture 60 is turned such that the aperture 60 does not cover the opening 21 and the additional air is allowed to flow through the entire cross-section of the opening 21. Thus, during this first position the maximum additional air is supplied to the suction channel 18, so that the lower limit value of the underpressure adjusts itself. If the rotary unit 52 and thus the aperture 60 are turned clockwise in direction of the arrow P2, the aperture 60 increasingly covers, with an increasing angle of rotation relative to the first position, the opening 21, so that the flow cross-section being provided for the additional air decreases, resulting in the volume flow of the additional air decreasing as well. As a result, a higher underpressure adjusts itself. In the second position shown in FIG. 5, the rotary unit 52, and thus the aperture 60, are turned so far that then the aperture 60 partly covers the opening 21 due to the larger distance B of the contour 68 of the aperture 60 to the centre axis 54.

If the aperture 60 is turned further in direction of the arrow P2, it entirely covers the opening 21 in the third position shown in FIG. 6, so that additional air is no longer allowed to flow through the opening 21 and the upper limit value of the underpressure adjusts itself.

Due to the triangular form of the opening 21 and the helical form of the aperture 60 it is achieved that there is a linear connection between the angle of rotation of the aperture 60 and the underpressure adjusting itself in the suction channel 18. Thus, during a turning of the aperture 60 by the same angle, the underpressure is in each case varied by the same value, so that in case of a respective synchronization between the ring 38 of the adjustment unit 26 and the rotary unit 52 the desired linear connection between the position of the adjustment unit 26 and the self-adjusting underpressure occurs.

Although various embodiments of the present invention have been described and shown, the invention is not restricted thereto, but may also be embodied in other ways within the scope of the subject-matter defined in the following claims.

What is claimed is:

1. A device for suction of liquids and particles from body orifices, comprising
a suction channel through which the liquid and/or the particles can be sucked,
a vacuum pump for generating an underpressure in the suction channel for suction of the liquid and/or the particles, wherein the suction channel is provided with an opening for supplying additional air for adjusting the underpressure,
an additional air adjustment unit for adjusting the volume flow of the additional air supplied to the suction channel, and
a manually operable adjustment unit for adjusting the additional air adjustment unit,
wherein at least in the range between a lower limit value of the underpressure and an upper limit value of the underpressure the adjustment of the adjustment unit in each case by a same preset adjustment way effects the same change in the underpressure , and
in that the adjustment unit is continuously adjustable.

2. The device according to claim 1, wherein the adjustment unit is stepwise adjustable and that, at least in the range between the lower limit value and the upper limit value, the adjustment of the adjustment unit in each case by one step effects in each case the same change in the underpressure.

3. The device according to claim 1 wherein the additional air adjustment unit comprises a valve.

4. The device according to claim 1, wherein the additional air adjustment unit comprises a cover plate for covering at least a portion of an additional air channel for supplying the additional air and/or at least a portion of the opening for supplying the additional air.

5. The device according to claim 1, wherein a manometer for displaying the respective current underpressure is provided.

6. The device according to claim 5, wherein the manometer comprises a circular-sectored scale and a pointer rotatably arranged around the centre of the circular-sectored scale for displaying the current underpressure on the scale, that the adjustment unit comprises a ring whose centre coincides with the centre of the scale, that the ring is rotatably arranged around its centre for adjusting the underpressure, that on the ring at least one marking is provided and that the adjustment unit is synchronized to the manometer such that at least in the range between the lower limit value and the upper limit value in each case that underpressure adjusts itself that is displayed by the marking of the ring on the scale.

7. The device according to claim 1, wherein the device comprises a suction hose by means of which at least a portion of the suction channel is formed.

8. The device according to claim 1, wherein a motor is provided for driving the vacuum pump.

9. The device according to claim 1, wherein the upper limit value of the underpressure has a value between −70 kPa and −90 kPa, in particular a value of −80 kPa.

10. The device according to claim 1, wherein the lower limit value of the underpressure has a value between −0 kPa and −10 kPa.

11. The device according to claim 6, wherein the marking is formed as a deepening in the surface of the ring.

12. The device according to claim 6, wherein at least the visible surface of the ring has a first colour and that the marking has a second colour that is different from the first colour.

13. The device according to claim 6, wherein the ring is rotatable about an angle in the range between 90° and 270°, preferably by an angle of approximately 210°.

14. The device according to claim 1, wherein the opening and/or the additional air channel have a triangular cross-section, and that the additional air adjustment unit has a helical aperture for limiting the cross-section.
